(19) United States
(12) Patent Application Publication
(10) Pub. No.: US 2007/0077215 A1
(43) Pub. Date: Apr. 5, 2007

(54) Administration of phenylfurymethylthiazolidine-2,4-dione or phenylthienymethylthiazolidine-2,4-dione compounds for promoting and/or inducing and/or stimulating the pigmentation of keratinous substances and/or limiting their depigmentation and/or their whitening

(75) Inventors: Christophe Boulle, Paris (FR); Maria Dalko, Gif S/Yvette (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/543,194

(22) Filed: Oct. 5, 2006

Related U.S. Application Data
(60) Provisional application No. 60/726,206, filed on Oct. 14, 2005.

(30) Foreign Application Priority Data
Oct. 5, 2005 (FR) 05/53018

(51) Int. Cl. A61K 8/49; A61K 31/427

(52) U.S. Cl. 424/59; 514/369

(57) ABSTRACT

Phenylfurylmethylthiazolidine-2,4-dione and phenylthienylmethylthiazolidine-2,4-dione compounds of formula (1): 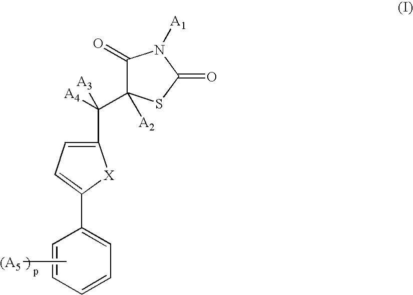 are useful for promoting and/or inducing and/or stimulating the pigmentation of keratinous substances and/or limiting their depigmentation and/or their whitening and more particularly for preventing and/or limiting canities of human keratinous fibers.

### CROSS-REFERENCE TO COMPANION APPLICATION

Copending U.S. patent application Ser. No. ______ [Attorney Docket No. 1016800-000783], filed concurrently herewith and assigned to the assignee hereof.

### CROSS-REFERENCE TO PRIORITY/PROVISIONAL APPLICATIONS

This application claims priority under 35 U.S.C. §119 of FR 05/53018, filed Oct. 5, 2005, and of U.S. Provisional Application No. 60/726,206, filed Oct. 14, 2005, each hereby expressly incorporated by reference and each assigned to the assignee hereof.

### BACKGROUND OF THE INVENTION

1. Technical Field of the Invention

The present invention relates to the administration of phenylfurylmethylthiazolidine-2,4-dione or phenylthienylmethylthiazolidine-2,4-dione compounds having specific formulae as bioactive agents for promoting and/or inducing and/or stimulating the pigmentation of keratinous substances or substrates and/or limiting their depigmentation and/or their whitening and more particularly as agent for preventing and/or limiting canities of human keratinous fibers.

The keratinous substances to which this invention relates comprise the skin, human nails and human keratinous fibers, such as the hair, eyebrows, eyelashes, beard hair or moustache hair. The present invention also relates to the keratinous substances of mammals of the animal type (dogs, horses or cats, for example). More specifically, this invention relates to human hair, beard hair, moustache hair, eyelashes and eyebrows.

2. Description of Background and/or Related and/or Prior Art

A need exists for novel products for caring for and/or treating human keratinous substances which promote their pigmentation and/or which limit their depigmentation and more particularly products which make it possible to prevent and/or reduce canities of human keratinous fibers, such as the hair, eyelashes and/or some non-head hair.

The color of human hair and of human skin depends on various factors and in particular on the seasons of the year, race, sex and age. It is mainly determined by the concentration of melanin produced by the melanocytes. The latter are specialized cells which synthesize melanin via specific organelles, the melanosomes.

The synthesis of melanin (or melanogenesis) is complex and schematically involves the following main stages:
tyrosine→dopa→dopaquinone→dopachrome→melanin

Tyrosinase (monophenol dihydroxyl phenylalanine: oxygen oxidoreductase EC 1.14.18.1) is involved in this sequence of reactions by catalyzing in particular the reaction for the conversion of tyrosine to dopa (dihydroxyphenylalanine) and the reaction for the conversion of dopa to dopaquinone.

The top part of the hair follicle exists as a tubular invagination of the epidermis which penetrates as far as the deep layers of the dermis. The bottom part, or hair bulb, itself comprises an invagination in which the dermal papilla is found. A region populated by cells with a high degree of proliferation (matrix cells) is found in the bottom part of the bulb, around the dermal papilla. These cells are the precursors of the keratinized cells of which an individual head hair will be composed. The cells which result from the proliferation of these precursors migrate vertically in the bulb and are gradually keratinized in the top part of the bulb; this assembly of keratinized cells will form the hair shaft. The pigmentation of the individual head hair and of non-head hairs requires the presence of melanocytes in the bulb of the hair follicle. These melanocytes are in an active state, that is to say that they are synthesizing melanins (or melanin pigments). These pigments are passed on to the keratinocytes intended to form the hair shaft, which will result in the growth of a pigmented individual head hair or of a pigmented individual non-head hair. This structure is referred to as “follicular pigmentation unit”.

It is known that, in the majority of populations, a brown skin coloration and the maintenance of a uniform coloration of the individual head hair are important aspirations.

It is accepted that the appearance of grey or white hair and/or non-head hairs, or canities, is associated with a decrease in melanin in the hair shaft. This phenomenon occurs naturally during the life of an individual. However, people are seeking to have a more youthful appearance and, for the purpose of attractiveness, they are often tempted to combat this phenomenon, in particular when it occurs at a relatively early age.

Numerous solutions have thus been provided in the field of artificial coloration by introducing exogenous dyes targeted at giving the hair a coloration as close as possible to that which it has naturally. Another approach consists in stimulating the natural pigmentation pathway.

Exemplary, among the solutions provided, are compositions comprising an inhibitor of phosphodiesterases (WO 95/17161), DNA fragments (WO 95/01773), diacylglycerol (WO 94/04122), prostaglandins (WO 95/11003) or pyrimidine 3-oxide derivatives (EP 8,292,60).

The assignee hereof has now found, unexpectedly, that it is possible to stimulate the synthesis of melanin by the melanocytes by specifically inhibiting the decomposition of the prostaglandins synthesized by these melanocytes or those present in their environment.

The involvement of certain prostaglandins in the pigmentation of non-head hairs or the skin in man or animals is described in the document Wand M., 1997, Arch. Ophtalmol., 115; Abdel Malek et al., 1987, Cancer Res., 47. However, as prostaglandins are molecules with a very short biological half-life and due to the local and labile nature of their metabolism (Narumiya S. et al.), it appears important to be able to prolong the activity of the prostaglandins involved in the pigmentation of human skin, hair and/or non-head hairs.

The assignee hereof has shown, in WO 04/073594, that 15-hydroxyprostaglandin dehydrogenase (15-PGDH) was also expressed in the hair melanocyte, which had never been demonstrated until then. In addition, the assignee hereof demonstrated the presence of 15-PGDH in the dermal papilla and the hair melanocyte and made provision for the use of a 15-PGDH inhibitor in order to promote the pigmentation of human skin, hair and/or non-head hairs. It is now possible to locally regulate the level of prostaglandins, in particular their presence in the melanocyte, especially the hair melanocyte, by acting on the decomposition catalyzed both by the 15-PGDH of the melanocyte and of the fibroblast of the dermal papilla.

Type-1 15-PGDH is a key enzyme in the deactivation of prostaglandins, in particular of PGF_{2α} and of PGE₂, which are important mediators of the growth and survival of the individual head hair. It corresponds to the classification EC 1.1.1.141 and is NAD⁺-dependent. This enzyme catalyzes an oxidation reaction of the hydroxyl on carbon 15 to give a ketone. It has been isolated from pig kidney; its inhibition has been observed in particular by a thyroid hormone, triiodothyronine, at doses much greater than physiological doses. For its part, type-2 15-PGDH is NADP-dependent.

In this same patent application, the assignee hereof had additionally shown that hair melanocytes expressed prostaglandin H synthase 1 (PGHS-1 or COX-1, EC: 1.14.99.1). This demonstrates for the first time that hair melanocytes have an autonomous prostaglandin metabolism.

Furthermore, it was shown, in WO 04/073594, that it was possible to specifically inhibit the 15-PGDH present in the dermal papilla and/or in the hair melanocyte. Such an inhibition thus makes it possible to slow down the deactivation of the prostaglandins in the environment of the hair melanocyte. The prostaglandins can thus continue, via an autocrine or paracrine route, to stimulate the melanocytes. Specifically, the application of such inhibitors stimulates the production of melanin by the melanocytes.

The same WO 04/073594 disclosed salified or non-salified phenylfurylmethylidenethiazolidine-2,4-dione compounds and phenylthienylmethylidenethiazolidine-2,4-dione compounds which have an inhibitory activity with regard to type-1 15-hydroxyprostaglandin dehydrogenase and which can be used as an agent for promoting and/or inducing and/or stimulating the pigmentation of human keratinous substances and/or as agent for preventing and/or limiting the depigmentation and/or the whitening of keratinous substances. However, the assignee hereof has found that these compounds exhibit the disadvantage of being colored and thus have a tendency, on application, to color keratinous substances and in particular keratinous fibers, the hair, eyebrows, eyelashes, beard hair or moustache hair.

According to the present invention, the term “15-PGDH inhibitor” means any substance, simple or complex compound, of natural or synthetic origin, capable of inhibiting or reducing the activity of the 15-PGDH enzyme and/or capable of inhibiting, reducing or slowing down the reaction catalyzed by this enzyme. The 15-PGDH inhibitors according to the invention are preferably inhibitors of type-1 15-PGDH.

Advantageously, the inhibitor is a specific inhibitor of NAD-dependent type-1 15-PGDH.

### SUMMARY OF THE INVENTION

It has now surprisingly been found that certain phenylfurylmethylthiazolidine-2,4-dione or phenylthienylmethylthiazolidine-2,4-dione compounds of formula (I), which will be defined in detail later, were inhibitors of 15-hydroxyprostaglandin dehydrogenase, in particular of type-1. It has also now been found that these same compounds promote and/or induce and/or stimulate the pigmentation of keratinous substances and limit their depigmentation and/or their whitening without the disadvantages of the compounds of the prior art.

The present invention thus features the cosmetic administration of at least one phenylfurylmethylthiazolidine-2,4-dione or phenylthienylmethylthiazolidine-2,4-dione compound of formula (I) or of one of its salts and/or of its solvates as bioactive agents for promoting and/or inducing and/or stimulating the pigmentation of keratinous substances and/or as agents for preventing and/or limiting the depigmentation and/or the whitening of keratinous substances and more particularly of human keratinous fibers, such as human hair, beard hair, moustache hair, eyelashes and eyebrows.

The present invention more particularly features the cosmetic administration of at least one phenylfurylmethylthiazolidine-2,4-dione or phenylthienylmethylthiazolidine-2,4-dione compound of formula (I) or of one of its salts and/or of its solvates as bioactive agents for preventing and/or limiting canities of human keratinous fibers.

Too, the present invention features the cosmetic administration of at least one phenylfurylmethylthiazolidine-2,4-dione or phenylthienylmethylthiazolidine-2,4-dione compound of formula (I) or of one of its salts and/or of its solvates in a care and/or makeup composition for inducing and/or stimulating the pigmentation of keratinous substances and/or limiting their depigmentation and/or their whitening and more particularly of human keratinous fibers, such as human hair, beard hair, moustache hair, eyelashes and eyebrows.

The present invention more particularly features the cosmetic administration of at least one phenylfurylmethylthiazolidine-2,4-dione or phenylthienylmethylthiazolidine-2,4-dione compound of formula (I) or of one of its salts and/or of its solvates in a care and/or makeup composition for preventing and/or limiting canities of human keratinous fibers.

This invention also features the cosmetic application of at least one phenylfurylmethylthiazolidine-2,4-dione or phenylthienylmethylthiazolidine-2,4-dione compound of formula (I) or of one of its salts and/or of its solvates in the formulation of compositions useful to induce and/or stimulate the pigmentation of keratinous substances and/or to inhibit their depigmentation and/or their whitening and more particularly of human keratinous fibers, such as human hair, beard hair, moustache hair, eyelashes and eyebrows.

The present invention more particularly features the application of at least one phenylfurylmethylthiazolidine-2,4-dione or phenylthienylmethylthiazolidine-2,4-dione compound of formula (I) or of one of its salts and/or of its solvates in the formulation of compositions useful to prevent and/or limit canities of human keratinous fibers, such as human hair, beard hair, moustache hair, eyelashes and eyebrows.

The present invention also features cosmetic processes, whether regime or regimen, for inducing and/or stimulating the pigmentation of keratinous substances and more particularly of human keratinous fibers and/or limiting their depigmentation and/or their whitening, which comprises topically applying, onto the said keratinous substances, an effective amount of at least one phenylfurylmethylthiazolidine-2,4-dione or phenylthienylmethylthiazolidine-2,4-dione compound of formula (I) or of one of its salts and/or of its solvates.

The present invention also features cosmetic regime or regimen for treating canities of human keratinous fibers, in particular of the hair, beard hair, moustache hair, eyelashes and/or eyebrows, which comprises topically applying, onto the said fibers, an effective amount of at least one phenylfurylmethylthiazolidine-2,4-dione or phenylthienylmethylthiazolidine-2,4-dione compound of formula (I) or of one of its salts and/or of its solvates.

The phenylfurylmethylthiazolidine-2,4-dione or phenylthienylmethylthiazolidine-2,4-dione compounds and their salts and/or their solvates in accordance with the invention have the following structural formula: 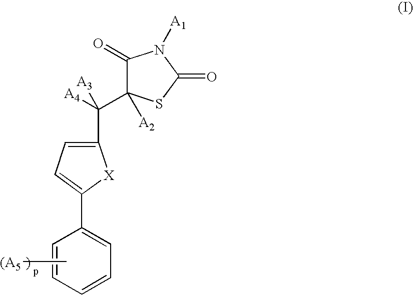
in which:
a) X is O or S;
b) A₁ is:

1) a hydrogen atom;

2) a linear or branched and saturated or unsaturated C₁-C₂₀ alkyl radical optionally substituted by one or more Z₁ groups;
c) the A₂, A₃, A₄ and A₅ radicals, which may be identical or different, are each:

1) a hydrogen atom;

2) a linear or branched and saturated or unsaturated C₁-C₂₀ alkyl radical optionally substituted by one or more Z₁ radicals;

3) a saturated or unsaturated hydrocarbon ring member having 3 to 7 atoms which is optionally substituted by one or more Z₁ groups;

4) a Z₁ radical;
d) p ranges from 0 to 5 inclusive and, in the event that p>1, the A₅ substituents can be identical or different;
e) Z₁ is:

1) a halogen, such as F, Cl or Br;

2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;

3) a linear or branched and saturated or unsaturated C₁-C₂₀ alkyl radical;

4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group, with the proviso that when A₅ is CH₂, Z₂ and Z′₂ are other than H and Me;
f) the Z₂, Z′₂, Z″₂ and Z′″₂ radicals independently are each:

1) a hydrogen atom;

2) a linear or branched and saturated or unsaturated C₁-C₂₀ alkyl radical optionally substituted by one or more Z₃ groups;

3) a saturated or unsaturated ring member having 4 to 15 atoms, optionally containing at least one heteroatom selected from among O, N or S, optionally fused to another ring, these rings optionally being substituted by one or more Z₃ groups; this ring is advantageously a phenyl;
g) the Z₃ radical is:

1) a Z₄ group;

2) a linear or branched and saturated or unsaturated C₁-C₂₀ alkyl radical optionally substituted by one or more Z₄ groups;

3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:

1) a halogen, such as F, Cl or Br;

2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅_{Z′″}₅, NZ₅SO₅Z′₅, COZ₅, COOZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:

1) a hydrogen atom;

2) a linear or branched and saturated or unsaturated C₁-C₂₀ alkyl radical;

3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

### DETAILED DESCRIPTION OF BEST MODE AND SPECIFIC/PREFERRED EMBODIMENTS OF THE INVENTION

The term “salts of compound of formula (I)” means, according to the invention, the organic or inorganic salts of a compound of formula (I).

Exemplary inorganic salts according to the invention are the sodium or potassium salts and the ammonium, zinc (Zn²⁺), calcium (Ca²⁺), copper (Cu²⁺), iron (Fe²⁺ and Fe³⁺), strontium (Sr₂₊), magnesium (Mg²⁺) and manganese (Mn²⁺) salts; hydroxides, hydrohalides (for example hydrochlorides), carbonates, hydrogencarbonates, sulfates, hydrogenphosphates or phosphates.

Exemplary organic salts according to the invention are salts of triethanolamine, monoethanolamine, ethanolamine, hexadecylamine and N,N,N′,N′-tetrakis(2-hydroxypropyl)ethylenediamine and those of organic acids, such as citrates, lactates, glycolates, gluconates, acetates, propionates, fumarates, oxalates and tartrates.

Exemplary solvates of the compounds of formula (I) are the hydrates, alcohol solvates or water/alcohol solvates.

According to the invention, the compounds of formula (I) are in the isolated or discrete form, that is to say non-polymeric form.

The term “at least one” according to the invention means one or more (2, 3 or more). In particular, the composition can comprise one or more compounds of formula (I). This or these compounds can be in the tautomeric form. This or these compounds can be enantiomers and/or diastereoisomers or a mixture of these isomers, in particular a racemic mixture.

The term “alkyl radical” means, according to the invention, a hydrocarbon radical which can be linear or branched and saturated or unsaturated. In particular, the alkyl radical has from 1 to 20 carbon atoms, preferably from 1 to 10. Examples of alkyl radicals according to the invention are the methyl, ethyl, isopropyl, n-butyl, tert-butyl, n-hexyl, 2-ethylhexyl, ethylene and propylene radicals.

Exemplary halogen atoms according to the invention are chlorine, fluorine and bromine atoms and preferably the chlorine and fluorine atoms.

Exemplary saturated ring members according to the invention are the cyclopropyl, cyclobutyl, cyclopentyl, cyclohexyl, cycloheptyl and adamantyl radicals.

Exemplary unsaturated ring members are the cyclohexenyl and phenyl radicals. Exemplary fused hydrocarbon rings of the invention are naphthyl and azulenyl radicals.

Exemplary fused rings of different nature according to the invention are the benzofuran, dibenzofuran, benzothiophene, benzothiazole, indole, benzimidazole, quinoline, isoquinoline, quinazoline, carboline, chromene, carbazole and fluorene radicals.

Exemplary ring members containing a carbonyl or thiocarbonyl functional group according to the invention are the following rings:

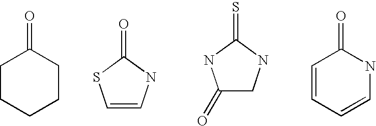

Exemplary heterocycles according to the invention are the azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine and crown ether 15-C-5 rings. Prefered are the pyrrole, pyrrolidine, imidazole, furan, thiophene, oxazole, thiazole, isoxazole, isothiazole, oxadiazole, pyrazole, tetrazole, pyridine, pyrimidine, triazole, pyrazine, pyridazine, piperidine, piperazine and morpholine ring members.

The preferred compounds of formula (I) in accordance with the invention are those for which the following conditions are satisfied:

(a) the A₁, A₂, A₃ and A₄ radicals simultaneously are hydrogen;

(b) p has the value 0, 1 or 2;

(c) the A₅ radical or radicals, which may be identical or different, are each a saturated or unsaturated and linear or branched C₁-C₁₀ alkyl radical optionally substituted by a Z₁ group or a Z₂ group;

(d) Z₁ is a halogen, CN, NO₂, CF₃, OZ₂, OCOZ₂, COOZ₂ or a phenyl ring optionally fused to another ring and optionally substituted by one or more Z₄ groups as defined above;

(e) Z₂ is hydrogen or a saturated or unsaturated and linear or branched C₁-C₁₀ alkyl radical.

Preferred compounds of formula (I) are the following and their salts and/or their solvates:

| Compound | Name |
|---|---|
| 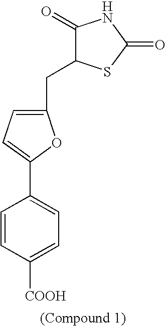 | 4-{5-[(2,4-Dioxo-1,3- thiazolidin-5-yl)methyl]-2- furyl}benzoic acid |
| 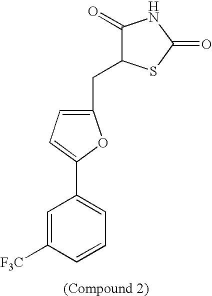 | 5-([5-[3- (Trifluoromethyl)phenyl]-2- furyl]methyl)-1,3-thiazolidine- 2,4-dione |
| 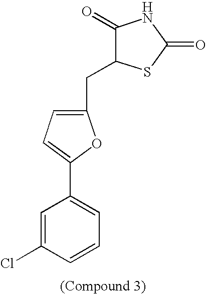 | 5-{[5-(3-Chlorophenyl)-2- furyl]methyl}-1,3-thiazolidine- 2,4-dione |
| 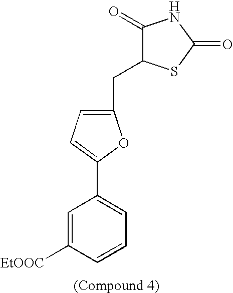 | Ethyl 3-{5-[(2,4-dioxo-1,3- thiazolidin-5-yl)methyl]-2- furyl}benzoate |
| 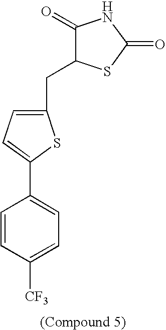 | 5-({5-[4- (Trifluoromethyl)phenyl]thien-2- yl}methyl)-1,3-thiazolidine-2,4- dione |
| 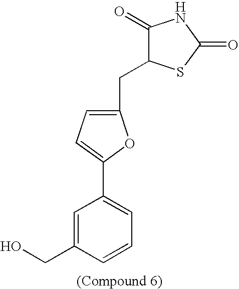 | 5-([5-[3- (Hydroxymethyl)phenyl]-2- furyl]methyl)-1,3-thiazolidine- 2,4-dione |
| 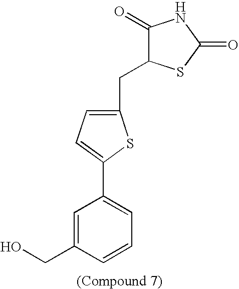 | 5-({5-[3- (Hydroxymethyl)phenyl]-2- thienyl}methyl)-1,3-thiazolidine- 2,4-dione |
| 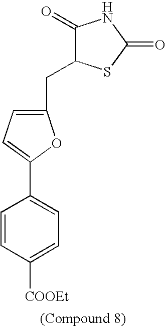 | Ethyl 4-{5-[(2,4-dioxo-1,3- thiazolidin-5-yl)methyl]-2- furyl}benzoate |
| 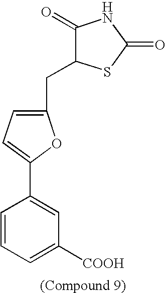 | 3-{5-[(2,4-Dioxo-1,3- thiazolidin-5-yl)methyl]-2- furyl}benzoic acid |
| 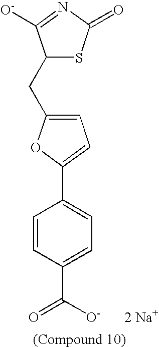 | Disodium salt of 4-{5-[(2,4- dioxo-1,3-thiazolidin-5- yl)methyl]-2-furyl}benzoic acid |
| 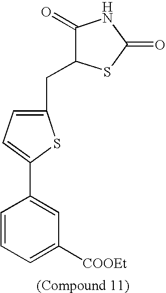 | Ethyl 3-{5-[(2,4-dioxo-1,3- thiazolidin-5-yl)methyl]-2- thienyl}benzoate |
| 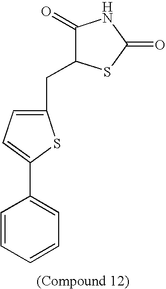 | 5-[(5-Phenyl-2-thienyl)methyl]- 1,3-thiazolidine-2,4-dione |
| 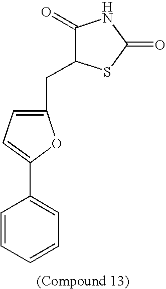 | 5-[(5-Phenyl-2-furyl)methyl]- 1,3-thiazolidine-2,4-dione |

More particularly preferred are:

4-{5-[(2,4-dioxo-1,3-thiazolidin-5-yl)methyl]-2-furyl}benzoic acid of formula:

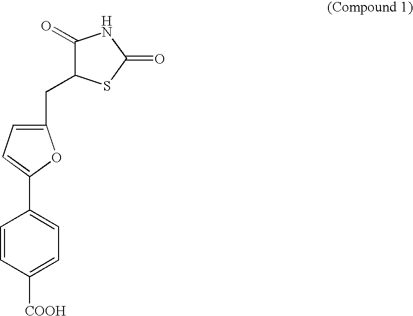

disodium salt of 4-{5-[(2,4-dioxo-1,3-thiazolidin-5-yl)methyl]-2-furyl}benzoic acid of formula: 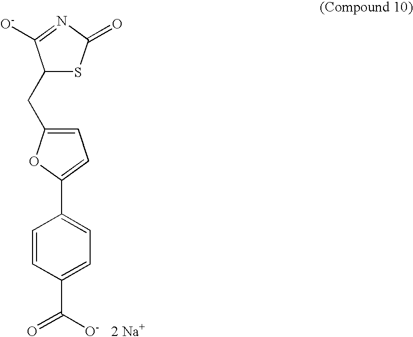
and their salts and/or their solvates.

The compounds of formula (I) in accordance with the invention can be prepared by a process for the reduction of a compound having the following structure: 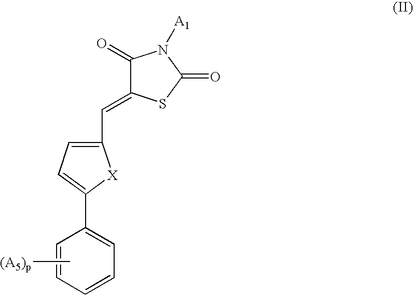
in which A₁ and A₅ and X and p have the same meanings as in the formula (I) defined above, and according to the following reaction scheme:

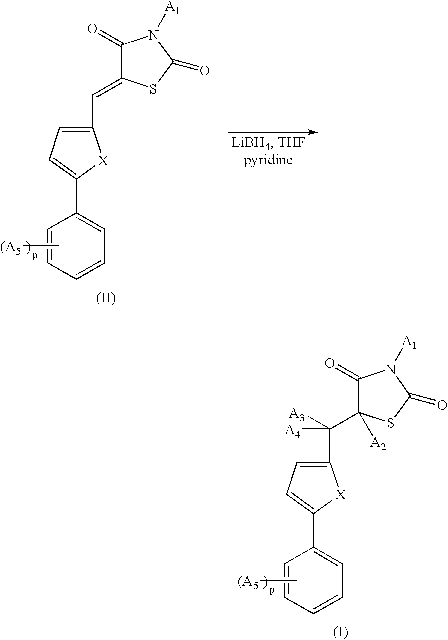

The compounds of formula (II) are disclosed, as is the synthesis thereof, in patent application Ser. No. 04/028441.

The effective amount of compound of formula (I) (salified or non-salified, solvated or non-solvated) corresponds to the amount of compound necessary to elicit the desired result or response (namely, to induce or stimulate keratinous pigmentation, in particular of the hair and eyelashes, and/or to reduce their pigmentation and/or their whitening). One skilled in this art is therefore in a position to evaluate this effective amount, which depends on the nature of the compound administered, on the individual to which it is applied and on the time of this application.

Hereinafter, unless otherwise indicated, the amounts of the various ingredients of the composition are given as percentage by weight with respect to the total weight of the composition.

To provide an order of magnitude, according to the invention, the compound of formula (I) (salified or non-salified, solvated or non-solvated) or a mixture of compounds of formula (I) (salified or non-salified, solvated or non-solvated) can be formulated in an amount ranging from 10⁻³% to 10% of the total weight of the composition and preferably in an amount ranging from 10⁻³% to 5% and better still from 10⁻²% to 2% of the total weight of the composition, for example from 0.5% to 2%.

The compositions of the invention can be for cosmetic or pharmaceutical applications. Preferably, the compositions of the invention are for cosmetic application. Consequently, the composition should comprise a physiologically acceptable medium which is non-toxic and which is capable of being topically applied to human skin, including the scalp and eyelids, and to human keratinous fibers. The term “cosmetic” means, according to the invention, a composition with a pleasant appearance, smell and feel.

The compounds of formula (I) (salified or non-salified, solvated or non-solvated) can be formulated in a composition which has to be ingested, injected or applied to the skin or to keratinous fibers (over any cutaneous region or all of the fibers to be treated).

According to the invention, the compounds of formula (I) or a mixture of compounds of formula (I) can be administered orally in an amount of 0.1 mg to 300 mg per day, in particular of 5 mg/day to 10 mg/day.

A preferred composition of the invention is a composition for cosmetic application and in particular for topical application to the skin and keratinous fibers and more especially to the scalp, hair and eyelashes.

This composition can be provided in any known dosage form which is suited to the method of administration.

For topical application to the skin or keratinous fibers, the composition can have the form of an aqueous, alcoholic or aqueous/alcoholic solution or suspension or of an oily suspension or solution, of an emulsion or dispersion with a more or less fluid consistency and in particular a liquid or semi-liquid consistency, obtained by dispersion of a fatty phase in an aqueous phase (O/W) or vice versa (W/O), of an (O/W) or (W/O) solid emulsion or dispersion, of an aqueous, aqueous/alcoholic or oily gel which is more or less fluid or solid, of a free or compact powder to be administered as is or to be incorporated in a physiologically acceptable medium, or also of microcapsules or microparticles, or of vesicular dispersions of ionic and/or nonionic type.

Also intended are compositions in the form of a foam or in the form of a spray or aerosol then comprising a pressurized propellant.

It can thus be provided in the form of a lotion, serum, milk, O/W or W/O cream, gel, ointment, pomade, powder, balm, patch, impregnated pad, cake or foam.

In particular, the compositions for application to the scalp or hair can be provided in the form of a hair care lotion, for example for daily or twice-weekly application, of a shampoo or of a hair conditioner, in particular for twice-weekly or weekly application, of a liquid or solid soap for cleaning the scalp, for daily application, of a product for shaping the hairstyle (lacquer, hairsetting product, styling gel), of a treatment mask, of a cream or of a foaming gel for cleaning the hair. It can also be provided in the form of a hair dye or mascara to be applied with a brush or comb.

Furthermore, for application to the eyelashes or non-head hair, the compositions to which this invention relates can be provided in the form of a pigmented or non-pigmented mascara, to be applied with a brush to the eyelashes or alternatively to beard or moustache hair.

For a composition for administration by injection, the composition can be provided in the form of an aqueous lotion or of an oily suspension. For administration by the oral route, the composition can be provided in the form of capsules, of granules, of syrups to be taken orally or of tablets.

According to a specific embodiment, the composition according to the invention is provided in the form of a hair cream or lotion, of a shampoo, of a hair conditioner, of a hair mascara or of a mascara for the eyelashes.

The amounts of the various constituents of the physiological medium of the composition according to the invention are those generally used in the fields under consideration. In addition, these compositions are prepared according to conventional methods.

When the composition is an emulsion, the proportion of the fatty phase can range from 2% to 80% by weight and preferably from 5% to 50% by weight with respect to the total weight of the composition. The aqueous phase is adjusted according to the content of fatty phase and of compound(s) (I) and according to the content of possible additional ingredients, in order to obtain 100% by weight. In practice, the aqueous phase represents from 5% to 99.9% by weight.

The fatty phase can comprise fatty or oily compounds which are liquid at ambient temperature (25° C.) and atmospheric pressure (760 mmHg), generally known as oils. These oils may or may not be compatible with one another and may form a macroscopically homogeneous liquid fatty phase or a two- or three-phase system.

The fatty phase can, in addition to the oils, comprise waxes, gums, lipophilic polymers, or “pasty” or viscous products comprising solid parts and liquid parts.

The aqueous phase comprises water and optionally an ingredient miscible in any proportion with water, such as lower C₁ to C₈ alcohols, for example ethanol or isopropanol, polyols, such as propylene glycol, glycerol or sorbitol, or else acetone or ether.

For a composition in the emulsion form, the composition can comprise one or more emulsifiers optionally in combination with one or more coemulsifiers used to produce a composition in the emulsion form. These emulsifiers and coemulsifiers are those generally used in the cosmetic and pharmaceutical fields. In addition, their nature depends on the sense of the emulsion. In practice, the emulsifier and optionally the coemulsifier are present in the composition in a proportion ranging from 0.1% to 30% by weight, preferably from 0.5% to 20% by weight and better still from 1% to 8%. In addition, the emulsion can comprise microcapsules or microparticles, vesicular dispersions and in particular lipid vesicles, such as liposomes.

When the composition is in the form of an oily solution or gel, the fatty phase can represent more than 90% of the total weight of the composition.

Advantageously, for a hair application, the composition of the invention is an aqueous, alcoholic or aqueous/alcoholic solution or suspension and better still a water/ethanol solution or suspension. The alcohol fraction can represent from 5% to 99.9% and better still from 8% to 80%.

For a mascara application, the composition of the invention is in particular in the form of a wax-in-water or wax-in-oil dispersion, of a gelled oil or of an aqueous gel, with or without pigment.

The compositions of the invention can comprise, in addition, other additional ingredients generally employed in the fields concerned and selected from among solvents, thickeners or gelling agents for the aqueous phase or for the oily phase, coloring materials which are soluble in the medium of the composition, solid particles of the filler or pigment type, antioxidants, sequestering agents, preservatives, fragrances, electrolytes, neutralizing agents, film-forming polymers, UV blocking agents, such as sunscreens, cosmetic and pharmaceutical active principles with a beneficial effect on the skin or keratinous fibers, other than the compounds of formula (I), or their mixtures. These additives can be present in the composition according to the amounts generally employed in the cosmetic and dermatological fields and in particular in a proportion of 0.01% to 50% of the total weight of the composition and better still of 0.1% to 20% and, for example, of 0.1% to 10%. These additives, depending on their nature, can be introduced into the fatty phase, into the aqueous phase and/or into the lipid vesicles and in particular liposomes.

Of course, one skilled in the art will take care to choose the possible additional ingredients and/or their amounts so that the advantageous properties of the composition according to the invention, namely the inhibition of 15-PGDH and in particular the increase in the density of keratinous fibers and/or the reduction in their whitening, are not, or not substantially, detrimentally affected by the envisaged addition.

Exemplary solvents according to the invention include lower C₂ to C₈ alcohols, such as ethanol or isopropanol, propylene glycol and certain light cosmetic oils, such as C₆ to C₁₆ alkanes.

Exemplary oils according to the invention are oils of mineral origin (liquid petrolatum, hydrogenated isoparaffin), oils of vegetable origin (liquid fraction of shea butter, sunflower oil, apricot oil, fatty alcohol or fatty acid), oils of animal origin (perhydrosqualene), synthetic oils (fatty acid esters, purcellin oil), silicone oils (phenyltrimethicone, linear or cyclic polydimethylsiloxane) and fluorinated oils (perfluoropolyethers). Exemplary waxes are silicone waxes, beeswax, rice wax, candelilla wax, carnauba wax, paraffin wax or polyethylene wax.

Exemplary emulsifiers according to the invention include glyceryl stearate or laurate, sorbitol stearates or oleates, alkyl dimethicone copolyols (with alkyl ≧8) and their mixtures for a W/O emulsion. Also exemplary are polyethylene glycol monostearate or monolaurate, polyoxyethylenated sorbitol stearate or oleate, dimethicone copolyols and their mixtures for an O/W emulsion.

Exemplary hydrophilic gelling agents according to the invention include carboxyvinyl polymers (carbomer), acrylic copolymers, such as acrylate/alkylacrylate copolymers, polyacrylamides, polysaccharides, such as hydroxypropylcellulose, natural gums and clays and exemplary lipophilic gelling agents include modified clays, such as bentones, metal salts of fatty acids, such as aluminum stearates, hydrophobic treated silica, ethylcellulose or their mixtures.

Exemplary cosmetic or pharmaceutical active principles other than the compounds of formula (I) according to the invention include hydrophilic active principles, such as proteins or protein hydrolysates, amino acids, polyols, urea, allantoin, sugars and sugar derivatives, water-soluble vitamins, plant extracts (those of Iridaceae or of soya) and hydroxy acids, such as fruit acids or salicylic acid; and lipophilic active principles, such as retinol (vitamin A) and its derivatives, in particular ester (retinol palmitate), tocopherol (vitamin E) and its derivatives, in particular ester (tocopherol acetate), essential fatty acids, ceramides, essential oils, salicylic acid derivatives, such as 5-(n-octanoyl)salicylic acid, esters of hydroxy acids, phospholipids, such as lecithin, or their mixtures.

Advantageously, the compositions according to the invention additionally comprise at least one prostaglandin or one prostaglandin derivative, such as, for example, series 2 prostaglandins, including in particular PGF_{2α}, and PGE₂, in the salt or ester form (for example, isopropyl esters), their derivatives, such as 16,16-dimethyl-PGE₂, 17-phenyl-PGE₂, 16,16-dimethyl-PGF_{2α}, 17-phenyl-PGF2α, series 1 prostaglandins, such as 11-deoxyprostaglandin E₁, 1-deoxyprostaglandin E₂, in the salt or ester form, their analogues, in particular latanoprost, (5E)-7-{(1R,2R,3R,5S)-3,5-dihydroxy-2-[(3R)-3-hydroxy-5-phenylpentyl]cyclopentyl}hept-5-enoic acid, viprostol, bimatoprost, cloprostenol, travoprost, fluprostenol, butaprost, unoprostone, misoprostol, their salts or their esters.

Also intended are compositions wherein at least the compound of formula (I), salified or non-salified, solvated or non-solvated is encapsulated, in particular in the liposome form, such as disclosed in particular in WO 94/22468. Thus, the encapsulated compound can be delivered selectively to the hair follicle.

The compositions according to the invention can be topically applied onto the regions of the skin to be treated and in particular to the alopecic regions of the scalp and of the hair of an individual, or only to white hair, and optionally maintained there in contact for several hours and optionally rinsed out.

It is possible, for example, to apply the composition comprising an effective amount of a compound of formula (I), salified or non-salified, solvated or non-solvated, in the evening, to maintain the composition in contact overnight and optionally to shampoo in the morning. These applications can be repeated daily for one or more months, depending on the individual.

Advantageously, in the regime or regimen according to the invention, from 5 μl to 10 ml of a solution or composition as defined above, comprising from 0.001% to 5% of 15-PGDH inhibitor, are applied to the regions of the scalp and/or of the hair to be cared for or treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Synthesis of 5-[5-(3-(trifluoromethyl)phenyl)fur-2-ylmethyl]-1,3-thiazolidine-2,4-dione (compound 2)

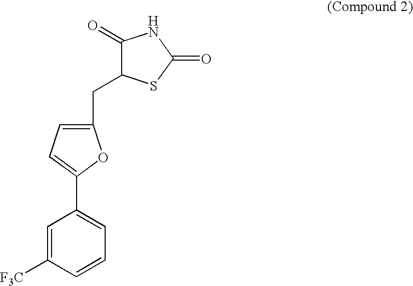

500 mg (1.47 mmol, 1 eq.) of 5-[5-(3-(trifluoromethyl)phenyl)fur-2-ylmethylene]-1,3-thiazolidine-2,4-dione are dissolved in 2 ml of anhydrous THF in a three-necked flask under a stream of argon and 2 ml of pyridine over KOH are added. 1.71 ml of lithium borohydride are added very gently (effervescence and increase in temperature). The reaction medium is heated at 65° C. for 3 hours. The mixture is homogeneous. The medium is left at ambient temperature and then it is poured slowly onto a cooled 1N hydrochloric acid solution. The medium is then extracted twice with ethyl acetate. The combined organic phases are washed with water, dried with sodium sulfate, filtered and concentrated as much as possible.

The residue obtained is purified by chromatography on silica gel (eluant:dichloromethane). The expected compound is obtained in powder form.

Results:

Appearance: light beige powder

Weight=84 mg

Yield=17%

Melting point=138° C.

Molecular weight=341 g.mol⁻¹

Analyses:

The NMR and mass spectra are in accordance with the expected structure.

### Example 2

### Synthesis of 5-{[5-(3-chlorophenyl)-2-furyl]methyl}-1,3-thiazolidine-2,4-dione (compound 3)

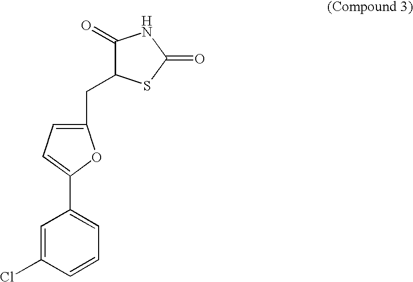

The procedure is identical to that of Example 1. The residue is purified by chromatography on silica gel (eluant: dichloromethane). The expected compound is obtained in powder form.

Results:

Appearance: powder

Weight=114 mg

Yield=6%

Melting point=104° C.

Molecular weight=307 g.mol⁻¹

Analyses:

The NMR and mass spectra are in accordance with the expected structure.

Elemental Analyses:

| | | | | | |
|---|---|---|---|---|---|
| C | H | N | O | S | Cl |

Theory

| | | | | | |
|---|---|---|---|---|---|
| 54.60% | 3.30% | 4.60% | 15.60% | 10.40% | 11.50% |

Analysis

| | | | | | |
|---|---|---|---|---|---|
| 54.32% | 3.17% | 4.55% | 16.13% | 10.51% | 10.85% |

### Example 3

### Synthesis of 5-({5-[4-(trifluoromethyl)phenyl]thien-2-yl}methyl)-1,3-thiazolidine-2,4-dione (compound 5)

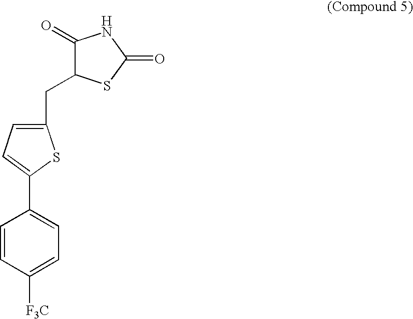

The procedure is identical to that of Example 1.

Purification is carried out on silica gel (eluant: heptane/ethyl acetate gradient: 80/20 to 50/50). The expected compound is obtained in powder form.

Results:

Appearance: light beige powder

Weight=79 mg

Yield=15%

Molecular weight=357 g.mol⁻¹

Analyses:

The NMR and mass spectra are in accordance with the expected structure.

### Example 4

### Synthesis of the Compound 4-[5-[(2,4-dioxo-1,3-thiazolidin-5-yl)methyl]-2-furyl}benzoic acid (compound 1)

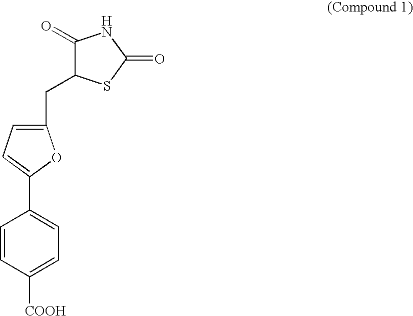

The procedure is identical to that of Example 1.

Analyses:

The NMR and mass spectra are in accordance with the expected structure.

### Example 5

### Synthesis of the Compound Disodium Salt of 4-{5-[(2,4-dioxo-1,3-thiazolidin-5-yl)methyl]-2-furyl)benzoic acid (compound 10)

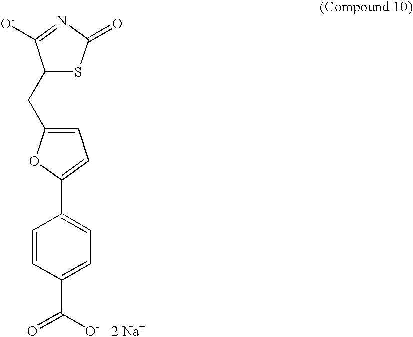

2.12 g of compound 1 are placed in 20 ml of water. 6.7 ml of a normal sodium hydroxide solution are added. The solid dissolves. After stirring for 30 minutes, the solution is poured onto acetone. A solid is formed, is filtered off and is then dried under vacuum. This solid, which comprises acetone, is dissolved in water and then brought to dryness. The residue is stirred in ethanol for 16 h, then filtered off and dried.

Appearance: powder

Weight=1.68 g

Yield=69%

Molecular weight=361 g.mol⁻¹

Analyses:

The NMR and mass spectra are in accordance with the expected structure.

### Example 6

### Demonstration of the Specific Inhibitory Properties of Compound 1 with respect to 15-PGDH

Test on Type-1 15-PGDH:

The enzyme 15-PGDH is obtained as disclosed in FR 02/05067, assigned to the assignee hereof, in suspension in a medium adjusted to a concentration of 0.3 mg/ml and then blocked at −80° C. For the requirements of the test, this suspension is defrosted and stored in ice.

Furthermore, a 100 mM, pH=7.4, Tris buffer comprising 0.1 mM of dithiothreitol (D5545, Sigma-Aldrich, L'isle D'Abeau Chesne, BP 701, 38297, Saint Quentin Fallavier) 1.5 mM of β-NAD (N6522, Sigma-Aldrich, L'isle D'Abeau Chesne, BP 701, 38297, Saint Quentin Fallavier) and 50 μM of prostaglandin E₂ (P4172, Sigma-Aldrich, L'isle D'Abeau Chesne, BP 701, 38297, Saint Quentin Fallavier) is prepared.

0.965 ml of this buffer (adjusted beforehand to 37° C.) is introduced into the cell of a spectrophotometer (Perkin-Elmer, Lambda 2) thermostatically controlled at 37° C., the measuring wavelength of which is adjusted to 340 nm. 0.035 ml of enzymatic suspension at 37° C. is introduced into the cell concomitantly with the recording (corresponding to an increase in the optical density at 340 nm). The maximum reaction rate is recorded.

The test values (comprising compound 1) are compared with the control value (without compound 1); the results shown represent either the percentage of inhibition of the enzymatic activity of 15-PGDH for a given concentration of compound of formula (I) or the concentration at which compound 1 reduces the enzymatic activity of 15-PGDH by 50%, namely IC_{50dh}.

The results are reported in the Table below.

| Compound | IC_{50dh} | |
|---|---|---|
| 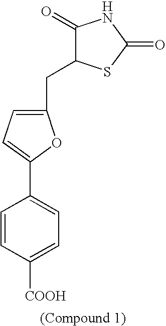 | 3.0 | μM |
| 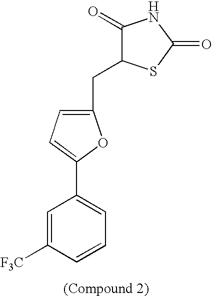 | 8.2 | μM |
| 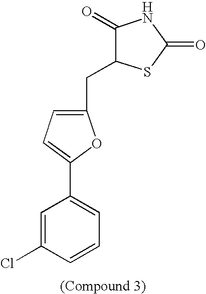 | 5.2 | μM |
| 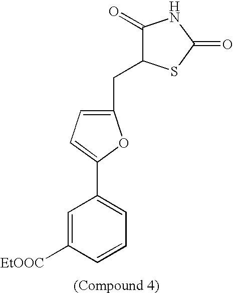 | 4.6 | μM |
| 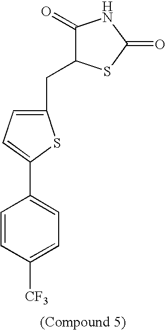 | 17.7 | μM |
| 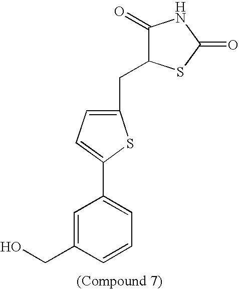 | 37.6 | μM |

It will thus be seen that compounds 1 to 4, 5 and 7 according to the invention are inhibitors of type-1 15-PGDH.

### Example 7

### Comparative Tests from a Compound of the Prior Art (WO 04/028441) and its Reduced Homologue According to the Invention

Hair Used:

The hair used for this experiment is of SA 40 grade (strong bleaching). Locks of hair weighing approximately 1 g are prepared and are washed with 0.4 g of shampoo. They are rinsed, dried with a hair dryer and then disentangled.

Protocol for Diluting the Products:

100 mg of product are weighed out and are treated with 3 ml of water, 1 ml of ethanol and 0.5 ml of a 20% aqueous ammonia solution (the medium being basic, the two compounds tested are thus in the salt form in the preparation medium). Stirring is maintained for 10 minutes. The lock is placed in a crystallizing dish without overlapping and then the solution is deposited over the lock using a pipette in order for the lock to be completely impregnated. The crystallizing dish is closed with a watchglass and then placed on a heating plate at 40° C. for 1 hour. The lock is subsequently superficially dried in absorbent paper and then disentangled and dried with a hair dryer.

Compounds Tested:

Ionic compound derived from 4-{5-[(2,4-disulfo-1,3-thiazolidin-5-ylidene)methyl]-2-furyl}benzoic acid (Z isomer) according to WO 04/028441 of formula:

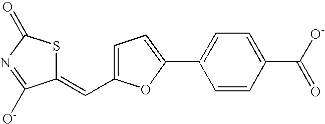

Compound according to the invention

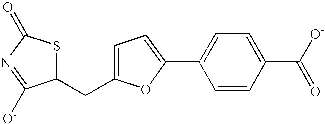

Control: the same operation is carried out without product.

It is found that the compound according to the prior art colors the locks of hair, in contrast to the compound according to the invention.

The compositions below are formulated by the usual techniques commonly employed in the cosmetic or pharmaceutical field.

### Example 8

Hair Lotion:

| | | |
|---|---|---|
| Compound 1 | 1.00 | g |
| Propylene glycol | 30.00 | g |
| Ethyl alcohol | 40.00 | g |
| Water | q.s. for 100.00 | g |

This lotion is applied to the scalp, once or twice daily, at the rate of 1 ml per application, the scalp being gently massaged in order to bring about the penetration of the active principle. The hair is subsequently dried in the open air. This lotion makes it possible to prevent and/or reduce canities of the hair.

### Example 9

Hair Lotion:

| | | |
|---|---|---|
| Compound 1 | 1.00 | g |
| Propylene glycol | 30.00 | g |
| Ethyl alcohol | 40.00 | g |
| Water | q.s. for 100.00 | g |

This lotion is applied to the scalp, once or twice daily, at the rate of 1 ml per application, the scalp being gently massaged in order to bring about the penetration of the active principle. The hair is subsequently dried in the open air. This lotion makes it possible to prevent and/or reduce canities of the hair.

### Example 10

Wax/Water Mascara:

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound 1 | 1.00% |
| Black pigment | 5.00% |
| Preservative | q.s. |
| Water | q.s. for 100.00% |

This mascara is applied to the eyelashes like a conventional mascara with a mascara brush.

### Example 11

Hair Lotion:

| | | |
|---|---|---|
| Compound 1 | 0.10 | g |
| Latanoprost | 0.10 | g |
| Propylene glycol | 30.00 | g |
| Ethyl alcohol | 40.00 | g |
| Water | q.s. for 100.00 | g |

This lotion is applied to the scalp, once or twice daily, at the rate of 1 ml per application, the scalp being gently massaged in order to bring about the penetration of the active principle. The hair is subsequently dried in the open air. This lotion makes it possible to prevent and/or reduce canities of the hair.

### Example 12

Hair Lotion:

(5E)-7-{(1R,2R,3R,5S)-3,5-Dihydroxy-2-[(3R)-3-hydroxy-5-phenylpentyl]cyclopentyl}

| | | |
|---|---|---|
| hept-5-enoic acid | 0.10 | g |
| Compound 1 | 0.10 | g |
| Propylene glycol | 30.00 | g |
| Ethyl alcohol | 40.00 | g |
| Water | q.s. for 100.00 | g |

This lotion is applied to the scalp, once or twice daily, at the rate of 1 ml per application, the scalp being gently massaged in order to bring about the penetration of the active principle. The hair is subsequently dried in the open air. This lotion makes it possible to prevent and/or reduce canities of the hair.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for promoting and/or inducing and/or stimulating the pigmentation of human keratinous substances and/or for preventing and/or limiting the depigmentation and/or whitening of keratinous substances, comprising administering to a subject in need of such treatment, for such period of time as required to elicit the desired response, a thus effective amount of at least one phenylfurylmethylthiazolidine-2,4-dione or phenylthienylmethylthiazolidine-2,4-dione compound having the following structural formula (I) or salt and/or solvate thereof: 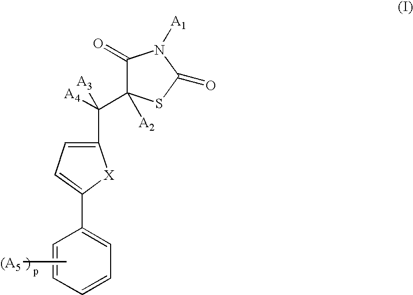
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p>1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

2. The regime or regimen as defined by claim 1, wherein formula (I), the following conditions are satisfied:
(a) the A₁, A₂, A₃ and A₄ radicals simultaneously are each hydrogen;
(b) p has the value 0, 1 or 2;
(c) the A₅ radical or radicals, which may be identical or different, is (are each) a saturated or unsaturated and linear or branched C₁-C₁₀ alkyl radical optionally substituted by a Z₁ group or a Z₂ group;
(d) Z₁ is a halogen, CN, NO₂, CF₃, OZ₂, OCOZ₂, COOZ₂ or a phenyl ring member optionally fused to another ring and optionally substituted by one or more Z₄ groups;
(e) Z₂ is hydrogen or a saturated or unsaturated and linear or branched C₁-C₁₀ alkyl radical.

3. The regime or regimen as defined by claim 1, said at least one compound of formula (I), or salt or solvate thereof, being selected from the group consisting of:

| Compound | Name |
|---|---|
| 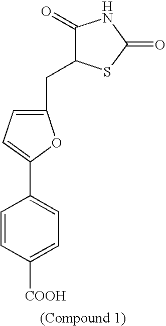 | 4-{5-[(2,4-Dioxo-1,3- thiazolidin-5-yl)methyl]-2- furyl}benzoic acid |
| 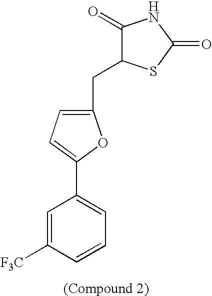 | 5-([5-[3- (Trifluoromethyl)phenyl]-2- furyl]methyl)-1,3-thiazolidine- 2,4-dione |
| 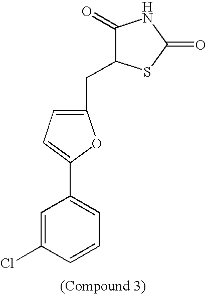 | 5-{[5-(3-Chlorophenyl)-2- furyl]methyl}-1,3-thiazolidine- 2,4-dione |
| 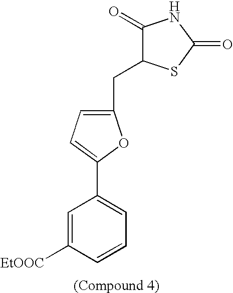 | Ethyl 3-{5-[(2,4-dioxo-1,3- thiazolidin-5-yl)methyl]-2- furyl}benzoate |
| 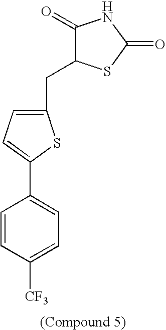 | 5-({5-[4- (Trifluoromethyl)phenyl]thien-2- yl}methyl)-1,3-thiazolidine-2,4- dione |
| 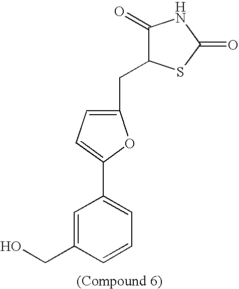 | 5-([5-[3- (Hydroxymethyl)phenyl]-2- furyl]methyl)-1,3-thiazolidine- 2,4-dione |
| 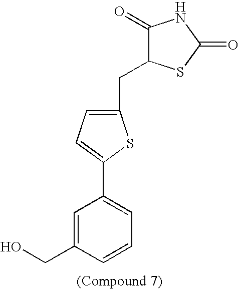 | 5-({5-[3- (Hydroxymethyl)phenyl]-2- thienyl}methyl)-1,3-thiazolidine- 2,4-dione |
| 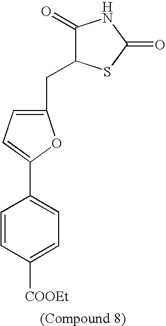 | Ethyl 4-{5-[(2,4-dioxo-1,3- thiazolidin-5-yl)methyl]-2- furyl}benzoate |
| 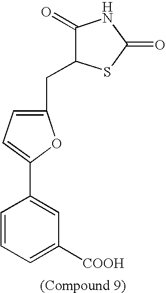 | 3-{5-[(2,4-Dioxo-1,3- thiazolidin-5-yl)methyl]-2- furyl}benzoic acid |
| 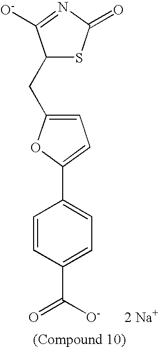 | Disodium salt of 4-{5-[(2,4- dioxo-1,3-thiazolidin-5- yl)methyl]-2-furyl}benzoic acid |
| 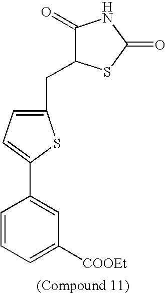 | Ethyl 3-{5-[(2,4-dioxo-1,3- thiazolidin-5-yl)methyl]-2- thienyl}benzoate |
| 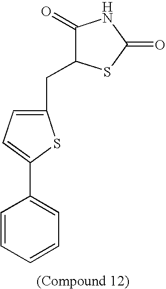 | 5-[(5-Phenyl-2-thienyl)methyl]- 1,3-thiazolidine-2,4-dione |
| 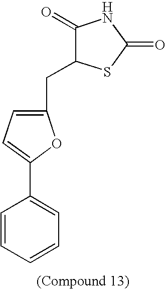 | 5-[(5-Phenyl-2-furyl)methyl]- 1,3-thiazolidine-2,4-dione. |

4. The regime or regimen as defined by claim 1, said at least one compound of formula (I), or salt or solvate thereof, being selected from the group consisting of 4-{5-[(2,4-dioxo-1,3-thiazolidin-5-yl)methyl]-2-furyl}benzoic acid of formula: 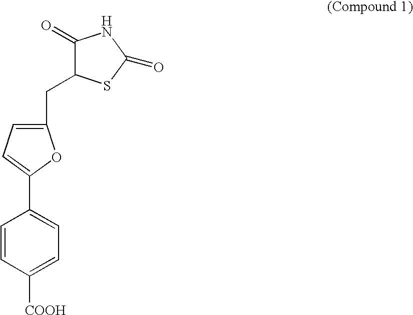
and the disodium salt of 4-{5-[(2,4-dioxo-1,3-thiazolidin-5-yl)methyl]-2-furyl}benzoic acid of formula:

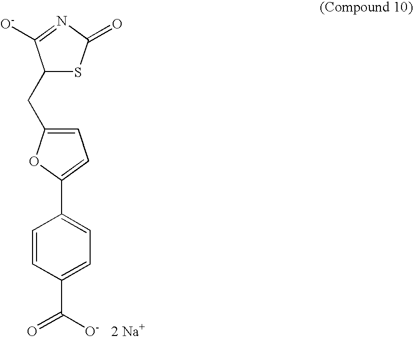

5. A regime or regimen for preventing and/or limiting canities of human keratinous fibers, comprising administering to an individual in need of such treatment, for such period of time as required to elicit the desired response, a thus effective amount of at least one phenylfurylmethylthiazolidine-2,4-dione or phenylthienylmethylthiazolidine-2,4-dione compound having the following structural formula (I) or salt and/or solvate thereof: 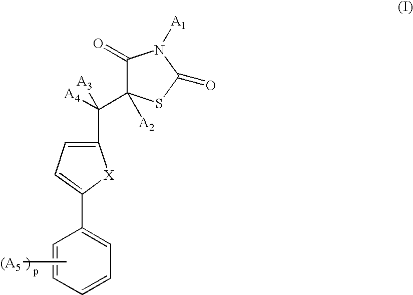
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p>1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅,CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

6. The regime or regimen as defined by claim 5, said human keratinous fibers comprising human hair, beard hair, moustache hair, eyelashes and/or eyebrows.

7. A regime or regimen for inhibiting 15-hydroxyprostaglandin dehydrogenase, comprising administering to a subject in need of such treatment, for such period of time as required to elicit the desired response, a thus effective amount of at least one phenylfurylmethylthiazolidine-2,4-dione or phenylthienylmethylthiazolidine-2,4-dione compound having the following structural formula (I) or salt and/or solvate thereof: 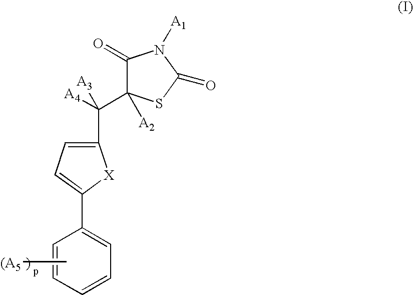
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p>1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

8. A regime or regimen for stimulating the production of melanin by the melanocytes, comprising administering to a subject in need of such treatment, for such period of time as required to elicit the desired response, a thus effective amount of at least one phenylfurylmethylthiazolidine-2,4-dione or phenylthienylmethylthiazolidine-2,4-dione compound having the following structural formula (I) or salt and/or solvate thereof: 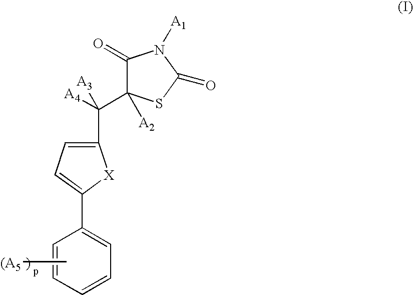
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p>1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁ -C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

9. The regime or regimen as defined by claim 1, comprising topically applying said at least one compound of formula (I), or salt or solvate thereof, onto the scalp, hair and/or eyelashes of a human subject.

10. A lotion, cream, serum, milk, emulsion, gel, ointment, pomade, powder, balm, patch, impregnated pad, cake, foam, spray, aerosol, shampoo, soap, mascara, makeup, lacquer, dye, hairsetting product, hair conditioner, syrup, capsules, tablets or granules comprising a 15-PGDH inhibiting amount of at least one compound having the following structural formula (I), or salt and/or solvate thereof: 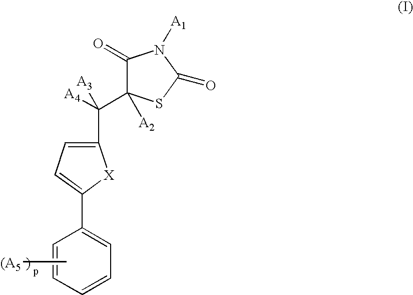
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p>1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

11. A composition of matter comprising at least one prostaglandin and/or derivative thereof and at least one compound having the following structural formula (I), or salt and/or solvate thereof: 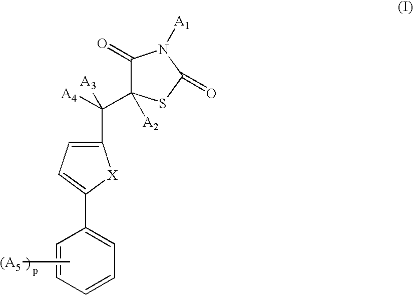
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p>1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ′₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅,CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.
